(19) United States
(12) Patent Application Publication
(10) Pub. No.: US 2025/0144179 A1
(43) Pub. Date: May 8, 2025

(54) PEPTIDE HORMONE TO PREVENT OR TREAT VASOSPASM, BRAIN INJURY OR CEREBRAL EDEMA

(71) Applicant: UNIVERSITY OF FLORIDA RESEARCH FOUNDATION, INCORPORATED, Gainesville, FL (US)

(72) Inventors: Brian Lim HOH, Gainesville, FL (US); Eduardo CANDELARIO-JALIL, Gainesville, FL (US); William S. DODD, Gainesville, FL (US); Koji HOSAKA, Newberry, FL (US)

(21) Appl. No.: 18/838,449

(22) PCT Filed: Feb. 16, 2023
(86) PCT No.: PCT/US2023/013201

§ 371 Date: Aug. 14, 2024

Related U.S. Application Data
(60) Provisional application No. 63/310,701, filed on Feb. 16, 2022.

(51) Int. Cl. A61K 38/22; A61K 9/00; A61P 7/04; A61P 25/00

(52) U.S. Cl. CPC A61K 38/22; A61K 9/0019; A61P 7/04; A61P 25/00

(57) ABSTRACT

Novel indication for adropin to prevent or treat vasospasm, brain injury, or cerebral edema.

### BACKGROUND

The pathophysiology of subarachnoid hemorrhage is complex. At ictus, a sharp rise in intracranial pressure decreases cerebral blood flow and induces a transient global ischemia. This early brain injury manifests as cerebral edema, blood-brain barrier (BBB) breakdown, microthrombosis, sympathetic nerve activation, and widespread apoptosis.¹ Over time the release of cell-free hemoglobin and heme into the subarachnoid space disrupts endothelial cell function by producing reactive oxygen species (ROS) and decreasing nitric oxide (NO) production.^{2,3} Diminished vasodilatory tone and activation of vasoconstrictive pathways is a hallmark of the delayed phase of subarachnoid hemorrhage (SAH) pathology and promotes the development of cerebral vasospasm and delayed cerebral ischemia (DCI).⁴

Cerebral endothelial dysfunction is a critical common element between the pathological mechanisms following subarachnoid hemorrhage.^{5,6} From BBB breakdown in the capillary beds, to microthrombosis in the small arteries and arterioles, to vasospasm of the large cerebral vessel, all areas of the cerebrovasculature are impacted by SAH. This evidence indicates that treatments exerting a vasoprotective effect will be most efficacious in treating SAH-related disease. Current treatments are only modestly effective⁷, reaffirming the need for novel therapeutic agents.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows SAH reduces adropin expression. FIG. 1A) relative mRNA expression in cultured mouse brain microvascular endothelial cells (BMvECs) exposed to cell-free hemoglobin (CFH), **p<0.01 by Student's t test. FIG. 1B) secreted adropin peptide concentration in BMvEC growth media after CFH exposure, *p<0.01 by Student's t test. FIG. 1C) Western blot of adropin in whole brain lysate in sham (left) and SAH (right) conditions 24 hours post-SAH in wild-type mice. Normalized densitometric analysis of adropin immunoblot, **p<0.01 by Student's t test.

FIG. 2 shows adropin preserves expression of tight junction proteins and decreases blood-brain barrier permeability. Occludin (FIG. 2A), ZO-1 (FIG. 2B), and MMP9 (FIG. 2C) immunoblots in wild-type mice treated with vehicle or adropin, *p<0.05 compared to sham+vehicle, #p<0.05 compared to SAH+vehicle by non-parametric Kruskal-Wallis test with Dunn's multiple comparisons. FIG. 2D) Blood-brain barrier permeability was measured in wild-type mice by sodium fluorescein extravasation (green) in sham+vehicle, SAH+vehicle, and SAH+adropin groups with semi-quantitative image analysis, **p<0.01 by non-parametric Kruskal-Wallis test with Dunn's multiple comparisons. FIG. 2E) Cerebral edema (brain water content) after treatment with synthetic adropin peptide, **p<0.01 by non-parametric Kruskal-Wallis test with Dunn's multiple comparisons.

FIG. 3 shows adropin treatment profoundly reduces microthrombi formation 24 hours after SAH. FIG. 3A) Representatives images of fibrinogen-immunostaining (red) in sham+vehicle (top left), SAH+vehicle (top right), and SAH+adropin (bottom left) groups, scale bars=50 μm, n=4 animals each group, **p<0.01 by non-parametric Kruskal-Wallis test with Dunn's multiple comparisons. FIG. 3B) Cerebral blood flow on day 1 post-SAH in sham+vehicle (top left), SAH+vehicle (top right), and SAH+adropin (bottom left). n=5-7 per group, *p<0.05 by non-parametrix Kruskal-Wallis test with Dunn's multiple comparisons.

FIG. 4 shows adropin reduces MCA vasospasm one-week post-SAH. FIG. 4A) Representative images of dye cast cerebrovasculature one-week post-SAH. Terminal interior carotid (ICA), anterior cerebral (ACA), and middle cerebral (MCA) arteries are labeled in sham images, scale bars=100 μm. FIG. 4B) Plot of minimum diameter along M1 segment, *p<0.05 by non-parametric Kruskal-Wallis test with Dunn's multiple comparisons. FIG. 4C) Bederson stroke scale scores, *p<0.05 by ordinary two-way ANOVA with Tukey's multiple comparisons. FIG. 4D) Corner-turn preference scores, *p<0.01 between SAH+vehicle and sham+vehicle groups, **p<0.01 between SAH+vehicle and sham+vehicle groups, & p<0.05 between SAH+adropin and sham+vehicle groups.

FIG. 5 shows adropin treatment reduces neuronal apoptosis one-week post-SAH. Representative images of TUNEL staining in sham+vehicle (left), SAH+vehicle (middle, and SAH+adropin (right) groups. Samples were co-stained with NeuN to differentiate between neuronal and non-neuronal cell populations and counterstained with DAPI, scale bars=50 μm.

FIG. 6 shows at one-day post-SAH, adropin has delivery-dependent effects on eNOS pathway: total eNOS is decreased by constitutive overexpression but preserved by exogenous adropin treatment. FIG. 6A) Representative immunoblots at 1-day post-SAH. Normalized densitometric values of phospho-eNOS (FIG. 6B), total eNOS (FIG. 6C) and p-eNOS/eNOS ratio (FIG. 6D). n=4 for all groups, *p<0.05, **p<0.01. E) Representative immunoblots at 7 days post-SAH. Normalized densitometric values for phospho-eNOS (FIG. 6F), total eNOS (FIG. 6G) and p-eNOS/eNOS ratio (H). n=4 each for all groups, *p<0.05.

FIG. 7 shows the protective effect of adropin treatment is abolished in eNOS knockout mice. FIG. 7A) Representative images of eNOS^{-/-} mice one-week post-SAH. Terminal interior carotid (ICA), anterior cerebral (ACA), and middle cerebral (MCA) arteries are labeled in sham images, scale bars=100 μm. FIG. 7B) Plot of minimum diameter along M1 segment, *p<0.05 by non-parametric Kruskal-Wallis test with Dunn's multiple comparisons. FIG. 7C) Bederson stroke scale scores, *p<0.05 by ordinary two-way ANOVA with Tukey's multiple comparisons. FIG. 7D) Corner-turn preference scores, p<0.01 at all time points between both SAH groups and sham+vehicle group.

FIG. 8 shows adropin maintains efficacy when treatment is delayed until 24 hours post-SAH. FIG. 8A) Representative images of mice one-week post-SAH. Terminal interior carotid (ICA), anterior cerebral (ACA), and middle cerebral (MCA) arteries are labeled in sham images, scale bars=100 μm. FIG. 8B) Plot of minimum diameter along M1 segment, *p<0.05 by non-parametric Kruskal-Wallis test with Dunn's multiple comparisons. FIG. 8C) Bederson stroke scale scores, *p<0.05 by ordinary two-way ANOVA with Tukey's multiple comparisons. FIG. 8D) Corner-turn preference scores, *p<0.01 between SAH+vehicle and sham+vehicle groups, & p<0.05 between SAH+adropin and sham+vehicle groups, #p<0.05 between SAH+vehicle and SAH+adropin groups.

FIG. 9 shows adropin increases symptom-free survival and decreases rupture symptomaticity following aSAH. FIG. 8A) Symptom-free survival curve after aneurysm induction, p=0.03 by Mantel-Cox log-rank test. FIG. 8B) Proportion of ruptured aneurysms that produced neurological symptoms or death. FIG. 8C) Aneurysm formation rates between vehicle and adropin treatments. FIG. 8D) Aneurysm rupture rates between vehicle and adropin treatments. Data from panels B-D analyzed with Fisher's exact test.

FIG. 10 shows adropin treatment is not overtly hepatotoxic. I.P. injection of adropin did not cause elevations in serum concentration of fasting glucose (top left), total bilirubin (top right), aspartate aminotransferase (AST) (bottom left), or alanine aminotransferase (ALT) (bottom right).

FIG. 11 shows mean arterial pressure (MAP) of mice before and after intracranial aneurysm induction. Adropin (blue line) and vehicle (red line) treatments had no effect on MAP before or after aneurysm induction, n=8-10 mice in each group at each time point, p>0.99 at all time points.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

The term “adropin” refers to a novel peptide hormone that regulates endothelial function and is highly expressed in the brain. Adropin is also referred to herein as active agent, unless indicated otherwise. It is involved in carbohydrate-lipid metabolism, metabolic diseases, central nervous system function, endothelial function, and cardiovascular disease. Current evidence suggests the primary action of adropin is stimulation of the eNOS pathway. Adropin, encoded by the energy homeostasis-associated (Enho) gene, was discovered in 2008 by Kumar et al. as a regulator of lipid metabolism and insulin sensitivity, highly expressed in liver and brain tissue.⁸ In one example, adropin pertains to the following sequence or a fragment thereof: SEQ ID NO: 1 CHSRSADVDSLSESSPNSSPGPCPEKAPPPQKPSHEGSYLLQP. Adropin may or may not also include the following signal peptide attached to a terminus thereof: SEQ ID NO: 2 MGAAISQGALIAIVCNGLVGFLLLLLWVILCWA. Reference to adropin may also include adropin functional variants.

“Functional variant” refers to a compound that possesses a biological activity (either functional or structural) that is substantially similar to the biological activity of adropin. The term “functional variant” is intended to include “variants” of adropin. The term “variant” is meant to refer to a molecule substantially similar in structure and function to adropin or a part thereof. A molecule is “substantially similar” to adropin if both molecules have substantially similar structures or if both molecules possess similar biological activity. A “functional variant” may include deletions (i.e., truncations) of one or more amino acid residues at the N-terminus or the C-terminus of a polypeptide disclosed herein; deletion and/or addition of one or more amino acid residues at one or more internal sites in the polypeptide disclosed herein; and/or substitution of one or more amino acid residues at one or more positions in the polypeptide disclosed herein.

In a specific embodiment, a functional variant comprises a fragment of Adropin having at least 25 contiguous amino acids of SEQ ID NO: 1. Accordingly, the inventive methods and compositions are likewise contemplated for functional variants of Adropin include peptides that are at least 50% identical (e.g., have at least 60%, 70%, 80%, 90%, 95% or more sequence identity) to SEQ ID NO: 1 and possess biological activity of adropin. In more specific embodiments, functional variants of adropin will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence protein sequence as disclosed herein, or any other specifically defined fragment of a full-length protein sequence as disclosed herein. Optionally, the variant polypeptides will have no more than one conservative amino acid substitution as compared to the native protein sequence, alternatively no more than 2, 3, 4, 5, 6, 7, 8, 9, or 10 conservative amino acid substitutions as compared to the native protein sequence.

As used herein, “identity” in reference to a nucleotide or amino acid sequence means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well known Smith Waterman algorithm can also be used to determine identity.

The term “brain injury” includes traumatic injuries and injuries as a result of disease, in particular injuries resulting in brain hemorrhage. Thus, “brain injury” includes, but is not limited to mild, moderate, or severe trauma to the brain such as that received in military conflict, sports injury, accidents and falls, and the like, and also includes but is not limited to injury to the brain as a result of any brain hemorrhage such as intracerebral hemorrhage or subarachnoid hemorrhage. In a specific embodiment, the brain injury is accompanied by, associated with, cerebral edema, vasospasm, blood brain barrier damage, or sensorimotor deficits.

The term “vasospasm” refers to a narrowing of the arteries in a subject.

The term “cerebral edema” refers to swelling of the brain of a subject. Cerebral edema is often caused by brain injury, strokes, brain tumor, infection and/or brain hemorrhage.

The term “brain condition” as used herein refers to brain injury, vasospasm and/or cerebral edema,

The term “dose” refers to a measured quantity of a medicine, nutrient, or pathogen which is delivered as a unit. A “unit dosage” refers to a unitary i.e. single dose, which comprises all the components of a composition of the disclosure, which is capable of being administered to a patient. A “unit dosage” may be readily handled and packed, remaining as a physically and chemically stable unit dose comprising the active agent and organic base with pharmaceutical carriers, excipients, vehicles, or diluents.

The term “therapeutically effective amount” relates to a dose of the substance that will lead to the desired pharmacological and/or therapeutic effect. The desired pharmacological effect is, to alleviate a condition or disease described herein, or symptoms associated therewith. A therapeutically effective amount of a substance may vary according to factors such as the disease state, age, sex, and weight of the individual, and the ability of the substance to elicit a desired response in the individual. Dosing regimen may be adjusted to provide the optimum therapeutic response. For example, several divided doses may be administered daily, or the dose may be proportionally reduced as indicated by the exigencies of the therapeutic situation.

The terms “endothelial nitric oxide synthase” or “eNOS” refer to an enzyme that in humans is encoded by the NOS3 gene located in the 7q35-7q36 region of chromosome 7. eNOS is primarily responsible for the generation of NO in the vascular endothelium, a monolayer of flat cells lining the interior surface of blood vessels, at the interface between circulating blood in the lumen and the remainder of the vessel wall. NO produced by eNOS in the vascular endothelium plays crucial roles in regulating vascular tone, cellular proliferation, leukocyte adhesion, and platelet aggregation. Therefore, a functional eNOS is essential for a healthy cardiovascular system.

The term, “pharmaceutically acceptable carrier” as used herein means a pharmaceutically-acceptable material, composition or vehicle, such as a liquid or solid filler, diluent, excipient, solvent or encapsulating material, involved in carrying or transporting the compositions of the invention from one organ, or portion of the body, to another organ, or portion of the body without affecting its biological effect. Each carrier should be “acceptable” in the sense of being compatible with the other ingredients of the composition and not injurious to the subject.

“Subject” means any animal, but is preferably a mammal, such as, for example, a human, monkey, non-human primate, mouse, or rabbit. A “subject in need” refers to a subject who has a brain condition.

The term “treating” or “treatment of” as used herein refers to providing any type of medical management to a subject. Treating includes, but is not limited to, administering a composition comprising one or more active agents to a subject using any known method. for purposes such as curing, reversing, alleviating, reducing the severity of, inhibiting the progression of, or reducing the likelihood of a brain condition or one or more symptoms or manifestations of a brain condition. The administration of the one or more active agents can be oral, nasal, parental, intracranial, topical, ophthalmic, or transdermal administration or delivery in the form of solid, semi-solid, lyophilized powder, or liquid dosage forms. The dosage forms include tablets, capsules, troches, powders, solutions, suspensions, suppositories, or the like, preferably in unit dosage forms suitable for simple administration of precise dosages.

### DETAILED DESCRIPTION

Embodiments of the present disclosure are based on the novel discovery that the peptide hormone adropin protects a subject following aneurysmal subarachnoid hemorrhage (aSAH). In a murine aSAH model, it was discovered that treatment with adropin reduced cerebral edema, preserved tight junction protein expression, and abolished microthrombosis at 1 day post-SAH. Adropin treatment also prevented delayed cerebral vasospasm, decreased neuronal apoptosis, and reduced sensorimotor deficits at seven days post-SAH. Delaying initial treatment of adropin until 24 hours post-SAH preserved the beneficial effect of adropin in preventing vasospasm and sensorimotor deficits. Mechanistically, adropin treatment increased eNOS phosphorylation (Ser1179) at one & seven days post-SAH. eNOS^{-/-} mice treated with adropin were not protected from vasospasm or behavioral deficits, indicating a requirement of eNOS in the adropin pathway. Finally, in a aneurysm rupture model, it was observed that adropin had no effect on aneurysm formation or rupture but treatment improved deficit-free survival and decreased rupture symptomaticity without altering systemic blood pressure.

In view of the surprising and medically beneficial findings outlined above, provided herein are methods for treating a brain condition in a subject in need by administering an thereapeutically effective amount of adropin or a functional variant thereof. The brain condition includes brain hemorrhage, intracerebral hemorrhage, subarachnoid hemorrhage (SAH), cerebral edema, BBB damage, or vasospasm, or sensorimotor deficits associated therewith. The adropin is typically administered with a pharmaceutically acceptable carrier. Administering may be conducted by a number of routes as will be deemed appropriate by trained medical personnel, including parenteral, intracranial, intraperitoneal, subcutaneous, intramuscular, or intravenous injection. The adropin will ideally be administered within 15 minutes to 24 hours of the brain condition occurring.

Additional embodiments are described in further detail below.

### Compositions, Dosing and Administration

Adropin is generally administered in an amount sufficient to treat or prevent a brain condition. In certain embodiments, adropin is provided in a pharmaceutical composition including a therapeutic amount of adropin effective to treat or prevent a brain condition. In certain embodiments, the pharmaceutical compositions comprises about 0.1 mg to 5 g of adropin.

In certain embodiments, the therapeutically effective amounts of adropin dose ranges from about 0.1 mg to 5 g. The therapeutic dose can vary widely for example from about 1-25 mg/day, 25-50 mg/day, 50-100 mg/day, 100-200 mg/day, 200-300 mg/day, 400-500 mg/day and 500-1000 mg/day, 0.5 mg to about 1 g, about 1 mg to about 750 mg, about 5 mg to about 500 mg, or about 10 mg to about 100 mg of adropin. The person of skill in the art, or any physician, is able to adjust the dose as needed and depending upon the patient's weight, general health, kidney and liver function, and the degree of disease being treated. Pentoxifylline has low toxicity, therefore considerably higher doses can be administered if necessary. The drug preferably is given orally, but also can be administered intravenously, subcutaneously, intramuscularly, intraperitoneally, intrathecally, transdermally, topically, intra-cranially, or by any other convenient means known in the art.

It is understood that the appropriate dose of an active agent depends upon a number of factors within the ken of the ordinarily skilled physician, veterinarian, or researcher for example, the identity, size, and condition of the subject or sample being treated, further depending upon the route by which the composition is to be administered, the type and strength of the formulation, the pharmacokinetics of the formulation, the frequency of administration, the severity of the disease, and the effect which the practitioner desires the an active agent to have. It is furthermore understood that appropriate doses of an active agent depend upon the potency with respect to the expression or activity to be modulated. Such appropriate doses may be determined using the assays described herein. When one or more of these active agents are to be administered to an animal (e.g., a human), a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Active agents can be administered as a single treatment or, preferably, can include a series of treatments that continue at a frequency and for duration of time that causes one or more symptoms of the enumerated disease to be reduced or ameliorated, or that achieves the desired effect including reducing tumor burden or metastasis. Typical frequencies of administration of therapeutic agents in embodiments of the invention include once per day, multiple times per day, every few days, every week or every few weeks, as needed and as determined by the physician. Active agents administered “together” can be administered at the same time in the same or different formulations, or at different times.

The term “administer” is used in its broadest sense and includes any method of introducing the compositions into a subject. Administration of an agent “in combination with” includes parallel administration of two agents to the patient over a period of time, co-administration (in which the agents are administered at approximately the same time, e.g., within about a few minutes to a few hours of one another), and co-formulation (in which the agents are combined or compounded into a single dosage form suitable for oral, topical, subcutaneous or parenteral administration).

Administration can also be intravenous, parenteral, intra-arterial, subcutaneous, intraperitoneal or intramuscular injection or infusion, intraopthalmic; or intracranial, e.g., intrathecal or intraventricular, administration. In recent years there has been a tendency towards the development of controlled release dosage forms that will provide therapy over an extended period of time. Normally this would be once a day, and it is believed that such a change in dosage regimen will reduce adverse reactions and side effects and also improve patient compliance. The use of synthetic polymers that may have muco-or bio-adhesive properties has been investigated and is disclosed in WO 85/02092.

In some embodiments, a slow-release preparation comprising the active agent is formulated. It is desirable to prolong delivery with these slow release preparations so that the drug may be released at a desired rate over this prolonged period. By extending the period, the drug can if required be released more slowly, which may lead to less-severe adverse reactions and side effects. The preparation of sustained, controlled, delayed or anyhow modified release form can be carried out according to different known techniques: 1. The use of inert matrices, in which the main component of the matrix structure opposes some resistance to the penetration of the solvent due to the poor affinity towards aqueous fluids; such property being known as lipophilia; 2. The use of hydrophilic matrices, in which the main component of the matrix structure opposes high resistance to the progress of the solvent, in that the presence of strongly hydrophilic groups in its chain, mainly branched, remarkably increases viscosity inside the hydrated layer; and 3. The use of bioerodible matrices, which are capable of being degraded by the enzymes of some biological compartment. See U.S. Pat. No. 7,431,943.

The term “slow release” refers to the release of a drug from a polymeric drug delivery system over a period of time that is more than one day wherein the active agent is formulated in a polymeric drug delivery system that releases effective concentrations of the drug. Drug delivery systems may include a plurality of polymer particles containing active drug material, each of the particles preferably having a size of 20 microns or less, and incorporating on the outer surface of at least some of the particles a bioadhesive material derived from a bacterium.

Such drug delivery systems have been described in U.S. Pat. No. 6,355,276. The use of these microorganisms in the design allow for a controlled release dosage form with extended gastrointestinal residence.

In certain embodiments, dosage forms of the compositions of the present invention include, but are not limited to, implantable depot systems.

Self-emulsifying microemulsion drug delivery systems (SMEDDS) are known in the art See U.S. Patent Application 2001/00273803. The term SMEDDS is defined as isotropic mixtures of oil, surfactant, cosurfactant and drug that rapidly form an oil-in-water microemulsion when exposed to aqueous media or gastrointestinal fluid under conditions of gentle agitation or digestive motility that would be encountered in the gastrointestinal tract.

Thermostable nanoparticles may be contained in a drug delivery system targeted for the GI tract. See U.S. Patent Application No. 2000/60193787. These drug delivery systems may include at least one type of biodegradable and/or bioresorbable nanoparticle and at least one drug that possesses at least one of the following properties: emulsifier or mucoadhesion. The drug may substantially cover the surface of the nanoparticle.

The therapeutic agent can be formulated with an acceptable carrier using methods well known in the art. The actual amount of therapeutic agent will necessarily vary according to the particular formulation, route of administration, and dosage of the pharmaceutical composition, the specific nature of the condition to be treated, and possibly the individual subject. The dosage for the pharmaceutical compositions of the present invention can range broadly depending upon the desired effects, the therapeutic indication, and the route of administration, regime, and purity and activity of the composition.

A suitable subject, preferably a human, can be an individual or animal that is suspected of having, has been diagnosed as having, or is at risk of developing a brain condition, and like conditions as can be determined by one knowledgeable in the art.

Techniques for formulation and administration can be found in “Remington: The Science and Practice of Pharmacy” (20th edition, Gennaro (ed.) and Gennaro, Lippincott, Williams & Wilkins, 2000), incorporated herein by reference.

A pharmaceutical compositions including adropin may be administered in a number of ways depending upon whether local or systemic treatment is desired and upon the area to be treated. Administration may be-oral, intraopthalmic, or parenteral. Parenteral administration includes intravenous, intraarterial, subcutaneous, intraperitoneal, or intramuscular injection or infusion; or intracranial, e.g., intrathecal or intraventricular, administration.

Active agent may be admixed, encapsulated, conjugated or otherwise associated with other molecules, molecule structures or mixtures of compounds, such as for example, liposomes, receptor targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Solutions or suspensions used for parenteral, intradermal, intraopthalmic, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens;

antioxidants such as ascorbic acid or sodium bisulfite; chelating agents such as ethylene diamine tetra acetic acid (EDTA); buffers such as acetates, citrates or phosphates; and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of the ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES® a glidant such as colloidal silicon dioxide; a sweetening agent such as sucrose or saccharin; or a flavoring agent such as peppermint, methyl salicylate, or orange flavoring.

Systemic administration can also be by transmucosal means to the buccal membrane, gums, intestine or colon. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

In one embodiment, the active agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) also can be used as pharmaceutically acceptable carriers. These can be prepared according to methods known to those skilled in the art.

### EXAMPLES

Provided below are descriptions of studies revealing underlying scientific discoveries in support of the embodiments herein.

### Example 1

### Methods and Materials

### Animals

All animal experiments were approved by IACUC (Institutional Animal Care and Use Committee) and performed in accordance with ARRIVE (Animal Research: Reporting of In Vivo Experiments) guidelines. All animals were 12-15-week-old females and were housed in pathogen-free housing with ad libitum food and water and 12-h light/dark cycling. Wild-type C57B1/6 mice were received from Charles River Labs (Wilmington, Mass.) and eNOS knockout mice were obtained from The Jackson Laboratory (Bar Harbor, Maine). eNOS knockout breeding pairs were genotyped prior to breeding and F1 progeny used for all experiments. The following primers were used for genotyping: eNOS knockout (Nos3^{-/-): F-}5′-AGGGGAACAAGCCCAGTAGT-3′, R-5′-CTTGTCCCCTAGGCACCTCT-3′. All animals underwent twice daily monitoring from institutional animal care services staff and/or investigators. Mice were randomized to treatment groups prior to surgical procedures.

### Pharmaceutics and Dosing

Synthetic adropin³⁴⁻⁷⁶ peptide was supplied from Bachem (Bubendorf, Switzerland) and diluted in 0.1% BSA in saline. 0.1% BSA in saline was used as vehicle control for all experiments. Adropin (450 nmol/kg) or an equivalent volume of vehicle was delivered via intraperitoneal injection 15 minutes post-SAH. For experiments ending at 24 hours post-SAH, an identical dose of adropin/vehicle was given again 12 hours later. For experiments ending 7 days post-SAH, adropin/vehicle was given again on days 1, 3 & 5 post-SAH. In the aneurysm rupture model, adropin was given at a dose of 450 nmol/kg starting 3 days before aneurysm induction (elastase injection). Adropin was given every other day until sacrifice

### Subarachnoid Hemorrhage Model

SAH was modeled using the anterior circulation single autologous blood injection method^{25,33} with minor adjustments. Mice were anesthetized with ketamine (100 mg/kg) and xylazine (10 mg/kg) before removing hair from scalp and cleaning with serial betadine—saline washes. The tail was also prepped from the base extending 1 cm distally. Mice were then fixed into a stereotaxic frame and body temperature maintained at 37° C. The scalp was incised along the sagittal suture from bregma to the nasal bone. The skin was reflected and a burr hole was drilled 5.0 mm rostral of bregma and 0.5 mm right laterally to avoid puncturing dural venous sinuses. Next, 150 μL of arterial blood was drawn from the ventral tail artery onto paraffin wax paper and hemostasis was achieved with manual pressure using sterile gauze. 50 μL of blood was drawn into a Hamilton syringe (Hamilton, Reno, Nevada) and passed through the cranial burr hole at an angle of 30° caudally until contact with the skull base (approximately 7.2 mm). The syringe was left in place for 30 seconds to allow for tissue accommodation before blood was manually injected at a rate of 10 μL/minute. After blood injection was complete, the syringe was left in place for an additional 3 minutes and then retracted slowly at a rate of 10 mm/minute. Sham surgery was the identical procedure and timing including placement of the stereotactic needle but without blood injection. After the needle and syringe were removed, the burr hole was covered and skin flap was sutured with 5-0 ethilon suture.

### Intracranial Aneurysm Rupture Model

Cerebral aneurysms were induced as previously described. Briefly, mice were anesthetized with ketamine (100 mg/kg) and xylazine (10 mg/kg) before the left common carotid artery and the right renal artery were ligated with 7-0 silk suture (Ethicon Inc., Somerville, New Jersey). Carotid and renal artery ligations are hereafter referred to as ligations. One week after ligations, mice were anesthetized and 10 μL of 1.0 U/mL porcine pancreatic elastase solution (Worthington Biochemical Corp, Lakewood, New Jersey) diluted in phosphate buffered saline (PBS, Invitrogen, Carlsbad, California) was stereotactically injected into the right basal cistern at 1.2 mm rostral of bregma, 0.7 mm lateral of midline, and 5.3 mm deep to the surface of the brain. A subcutaneous osmotic pump (Alzet, Cupertino, California) placed in the right flank immediately after elastase injection and continually infused angiotensin II (Bachem, Torrence, California) at a dose of 1000 ng/kg/min. Elastase injection and osmotic pump placement are hereafter referred to as aneurysm induction. Following recovery from aneurysm induction, mice were fed a diet of 8% NaCl with 0.12% beta-aminopropionitrile (BAPN; Harlan Laboratories, Indianapolis, Indiana).

Three weeks post-aneurysm induction, mice were deeply anesthetized with ketamine/xylazine. A bilateral anterolateral thoracotomy with transverse sternotomy was made to expose the heart and great vessels. The right atrium was punctured with the tip of a 23 g needle as an outlet before the left ventricle was sequentially perfused with normal saline, 4% paraformaldehyde (PFA), and Coomassie Brilliant Blue dye in a 20% gelatin solution. Brains were carefully removed from the skull and inspected under a dissection microscope for the presence of aneurysms as evidenced by outpouching of cerebral arteries in or connected to the circle of Willis. Aneurysm rupture was identified by the presence of extravasated blood near an identified aneurysm. Any mouse exhibiting stroke-like neurological symptoms (decreased motor activity, circling paresis, or ≥15% weight loss) less than three weeks post-aneurysm induction was immediately euthanized and brains inspected for evidence of hemorrhage or aneurysm rupture. Mice that died suddenly prior to the three weeks endpoint were inspected for evidence of intracranial hemorrhage and aneurysm rupture but were not included in histological studies.

### Immunofluorescence

Following perfusion-fixation and overnight post-fixation in 4% PFA, brain samples were embedded in paraffin wax and cut into 0.7 μm sections with a Leica RM2125 RTS microtome (Leica Microsystems, Buffalo Grove, New York) and adhered to poly-L-lysine-coated glass slides. Serial sections were deparaffinized in mixed xylenes (2×3 min, 27° C.), and rehydrated in graded ethanol solutions and deionized water. Slides were then washed with 1× Tris-buffered saline with 0.05% Tween-20 (TBST) before being incubated overnight with one or more of the following antibodies: rabbit anti-fibrinogen (ThermoFisher). Primary antibodies were detected using donkey anti-rabbit IgG AlexaFluor594, donkey anti-rabbit IgG AlexaFluor488, donkey anti-rat IgG AlexaFluor594, or donkey anti-goat IgG AlexaFluor488 (all from ThermoFisher, Waltham, Massachusetts). FITC-conjugated dUTP-based TUNEL kit was obtained from Abcam (Cambridge, Massachusetts) and performed according to manufacturer's instructions on NeuN-stained sections. Slides were mounted with a DAPI-containing antifade mounting medium (VectaShield, Vector Labs, Burlingame, California) and imaged using an Olympus IX71 fluorescent microscope (Olympus America, Center Valley, Pennsylvania). All images were analyzed by two blinded observers.

### Brain Water Content Measurements

Brain water content was measured using the wet weight—dry weight method.³⁴ Twenty-four hours post-SAH, brains were removed from the skull and the olfactory lobe and hindbrain were removed from the cortex and midbrain. Brains were weighed, placed into a 60° C. oven, and allowed to dry for 48 hours. Dried brain tissue was then reweighed and used to calculate the percent water mass.

### Sodium Fluorescein Extravasation Assay

Sodium fluorescein (NaFlu) extravasation was carried out as previously described.^{18,35} Twenty-four hours post-SAH, mice were injected with 100 μL of 2% NaFlu. After 10 minutes to allow for full circulation, mice were deeply anesthetized with ketamine/xylazine and perfused with 150 mL of ice-cold PBS to wash out all intravascular fluorescein. Working in the dark, brains were harvested, embedded into and fresh frozen before being cut into 0.7 μm coronal sections onto poly-L-lysine-coated slides. Slides were then mounted and imaged using an Olympus IX71 fluorescent microscope (Olympus America, Center Valley, Pennsylvania). Three images were taken per slide at random locations within the right cerebral cortex. Fluorescein positivity was normalized to a wild-type mouse that did not received NaFlu injection.

### Cerebral Blood Flow Measurements

One day post-SAH, animals were anesthetized with ketamine/xylazine as described above, placed in a supine position in a stereotaxic frame. The skull was exposed through the longitudinal sagittal incision and cleaned of any debris with a cotton-tipped applicator. A laser speckle contrast imager probe (LSCI, PeriMed, Jarfalla, Sweden) was positioned 10 cm above the skull surface such that the entire skull was in the field of view; the lambdoid suture caudally, frontonasal suture rostrally, and the temporalis muscles laterally. The LSCI sampled blood flow as a rate of 1 sample/second for two continuous minutes. Blood flow (perfusion) values are calculated using PIMsoft software (PeriMed AB, Stockholm, Sweden) as arbitrary “perfusion units”. The value for each animal was the average perfusion over the entire sampling period.

### Western Blotting

At 24 hours or 7 days post-SAH, mice were deeply anesthetized and perfused with 10 mL of ice-cold PBS. Brains were harvested from the skull and placed into a cold brain matrix (Harvard Apparatus, Holliston, Massachusetts). The olfactory lobe was removed, and brains were cut in half along the mid-sagittal plane. The right hemisphere was cut again 2 mm caudally from the rostral edge and the pieces of tissue were snap frozen in liquid N₂. Once all tissue samples were collected, brains were homogenized with a Dounce homogenizer and incubated in radioimmunoprecipitation assay (RIPA) buffer at 4° C. for 90 minutes with constant agitation. Samples were centrifuged, supernatant collected, and protein quantified via Bradford assay. Samples were then frozen at −20° C. or used immediately. 30 μg of total protein was separated electrophoretically through a 4-15% tris-glycine gel at 90V for 60 minutes and transferred onto nitrocellulose membranes with 0.45 μm pore size. Membranes were blocked with 5% skim milk in TBST (5% BSA for phospho-eNOS blots) for 1 hours at room temperature and then probed with specified primary antibodies (Table 1) diluted in 5% skim milk (5% BSA for phospho-eNOS) overnight at 4° C. with constant agitation. Membranes were then washed 3× with TBST and then incubated with HRP-conjugated secondary antibody diluted in 5% skim milk (5% BSA for phospho-eNOS) for 1 hour at room temperature with constant agitation. Finally, membranes were washed 5× with TBST and incubated with luminol-peroxide solution for 45 seconds before being exposed to film. Films were developed and densitometry performed using ImageJ software.

### Delayed Cerebral Vessel Casting

Middle cerebral artery diameter were measured as previously reported.^{36,37} One week post-SAH, mice were deeply anesthetized with ketamine and xylazine before being serially perfused through the left ventricle with PBS (5 mL), 4% PFA (15 mL), and 20% India ink dissolved in 5% gelatin. All solutions were warmed to 37° C. to prevent thermic effects on the vasculature and administered at a constant rate of 15 mL/min to mimic cardiac output.³⁸ Mouse carcasses were then stored at 4° C. overnight to allow for gelatin hardening. Brains were imaged and analyzed by an observer blinded to group assignment. The narrowest diameter within the first 2000 μm of the middle cerebral artery (MCA) after the terminal interior carotid artery bifurcation (M1 segment) was measured to assess vasospasm. Mice with anomalous cerebral vasculature (double MCA, bifurcated MCA, etc.) were excluded from data analysis.

### Statistical Analysis

All data are presented as mean±standard error of the mean (SEM) unless otherwise noted. No animals that underwent SAH procedure were excluded from data analysis for any reason. Comparisons of continuous variables between two groups were performed using the Mann-Whitney U test or Student's t test where appropriate. Non-parametric tests (Kruskal-Wallis with Dunn's multiple comparison) were used to compare differences of continuous variables among three or more groups. For neurobehavioral tests (composite neurological score & corner-turn test), we used a two-way ANOVA with Tukey's multiple comparison's test to determine the effect of group and time. For experiments in the aneurysm rupture model, Fisher's exact test was used to compare proportions of aneurysm formation, rupture, and rupture symptomaticity. Mantel-Cox (Log-rank) test was used to compare symptom-free survival. Power calculations were completed for the delayed vasospasm experiment with an a of 0.05, β of 0.2 to detect a minimum biological difference of 20% between groups and predicted standard deviation of 15% MCA diameter. All data analysis was performed using Prism data analysis software (GraphPad Software, San Diego, CA).

### Example 2

### Adropin Expression Decreases in Cultured Brain Endothelial Cells after Hemoglobin Exposure and in Murine Brain Tissue after SAH

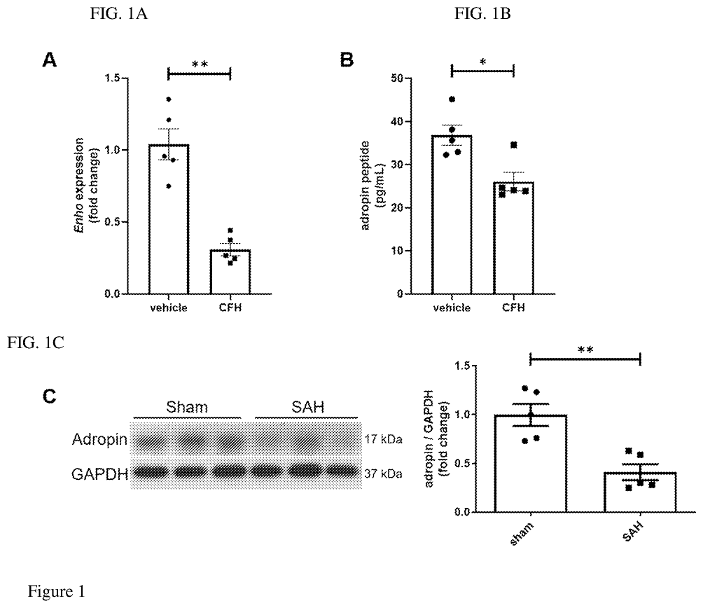

Little is known about the relationship between endogenous adropin production and cerebrovascular disease. It is highly expressed in wild-type mouse brain^{8,13} but role of disease state in modulating adropin expression remains to be investigated. Given adropin's protective role on endothelium, it was speculated that decreased adropin expression may be correlated with endothelial dysfunction following SAH. First, transcriptional & translational adropin expression was measured in isolated mouse brain microvascular endothelial cells (BMvECs) after simulated SAH (exposure to cell-free hemoglobin [CFH]). After 18 h of CFH exposure, Enho mRNA expression was decreased compared to vehicle-treated cells (FIG. 1A) as was secreted adropin peptide concentration (FIG. 1B). Next, immunoblotting was performed 24 hours post-SAH in the mouse model and found that adropin expression is markedly decreased in SAH mice compared to sham (FIG. 1C). The data suggest endogenous adropin expression falls within one day post-SAH and possibly due to the decrease in expression from the endothelial cells within the brain.

### Example 3

### Adropin Preserves Tight Junction Protein Expression And Decreases Cerebral Edema After SAH

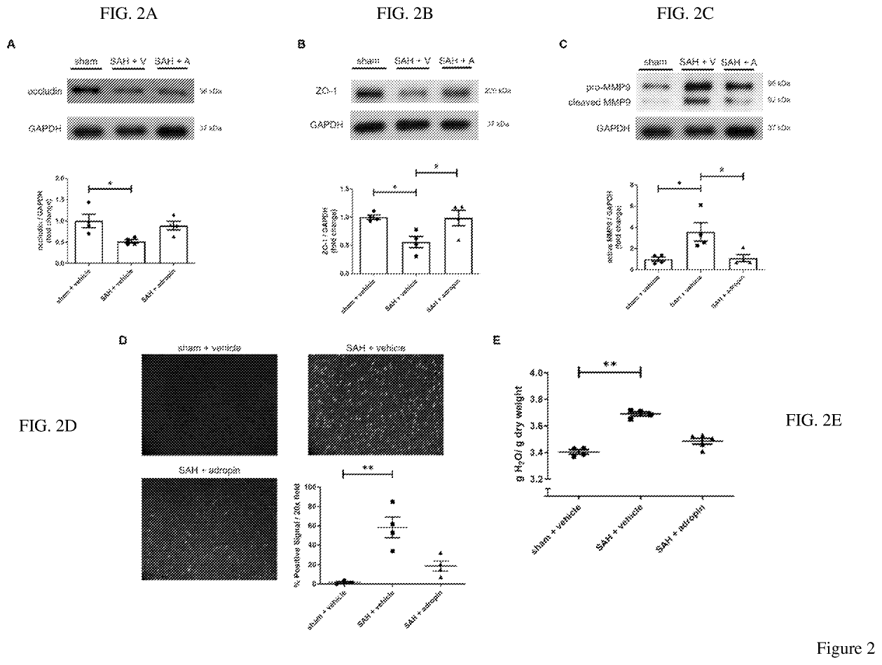

Tight junctions between adjacent endothelial cells are a critical component of the blood-brain barrier and help regulate fluid homeostasis, preventing cerebral edema. Many disease states are characterized by degradation of tight junctions by the protease MMP-9.^{14,15} The effect of adropin treatment on the tight junction protein occludin, tight junction-associated protein ZO-1, and MMP9 was investigated to further elucidate the effect of adropin on barrier function. Treatment with synthetic adropin also preserved the expression of occludin and ZO-1 after SAH compared to control (FIG. 2A&B). Consistent with this finding was the observation that SAH increased cleaved MMP9 levels compared to sham, an effect abolished by adropin treatment (FIG. 2C). Increased cerebral edema and BBB permeability, important predictors of mortality and neurological outcome, are downstream consequences of tight junction disruption.^{16,17} It was hypothesized that adropin would inhibit the development of cerebral edema and reduce BBB permeability. To investigate barrier function, a sodium fluorescein extravasation assay was used. Fluorescein does not bind plasma proteins, making it a sensitive marker for barrier disruption.¹⁸ SAH greatly increased barrier permeability as measured by average area of positive fluorescein signal compared to sham and adropin treatment reduced this effect (FIG. 2D). Similarly, SAH caused an increase in brain water content after SAH and this effect was blunted by adropin treatment (FIG. 2E). These data collectively suggest that adropin treatment prevents disruption of important blood-brain barrier structures and reduces permeability and edema.

### Example 4

### Adropin Treatment Abolishes Microthrombi Formation and Restores Cerebral Blood Flow after SAH

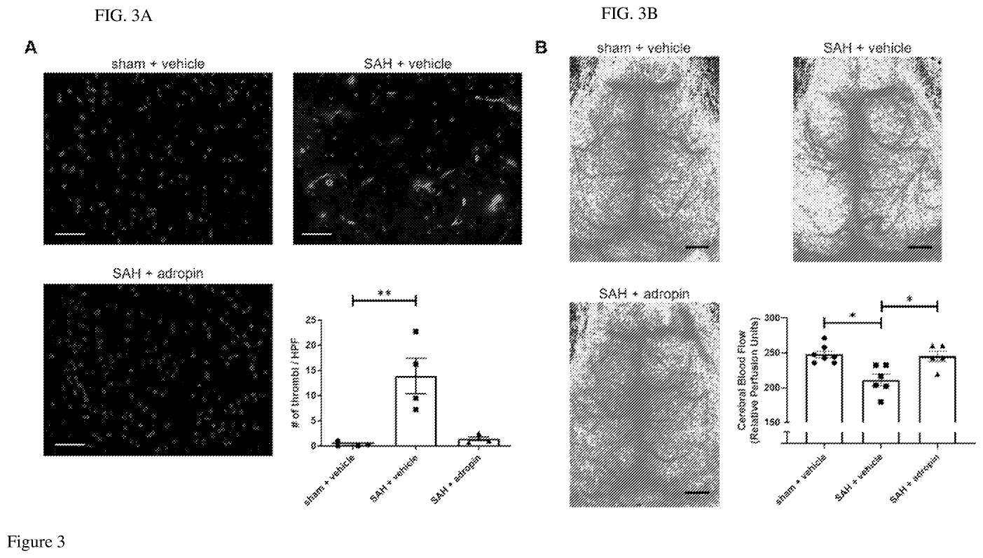

Increased microthrombosis within the cerebral arterioles and capillaries is a contributing factor towards reduced cerebral blood flow and development of delayed cerebral ischemia.¹⁹⁻²¹ Immunostaining for fibrinogen was performed 24 hours post-SAH in sham+vehicle, SAH+vehicle, and SAH+adropin mice to evaluate microthrombi formation. The SAH+vehicle group had an increased number of microthrombi compared to the sham group, however adropin treatment completely abolished this effect (FIG. 3A). Next, laser speckle contrast imaging was used to quantify cerebral blood flow (CBF) in mice after SAH. SAH induced a significant reduction in perfusion however, adropin treatment restored CBF back to baseline (FIG. 3B.)

### Example 5

### Adropin Attenuates Delayed Cerebral Vasospasm and Sensorimotor Deficits Following SAH

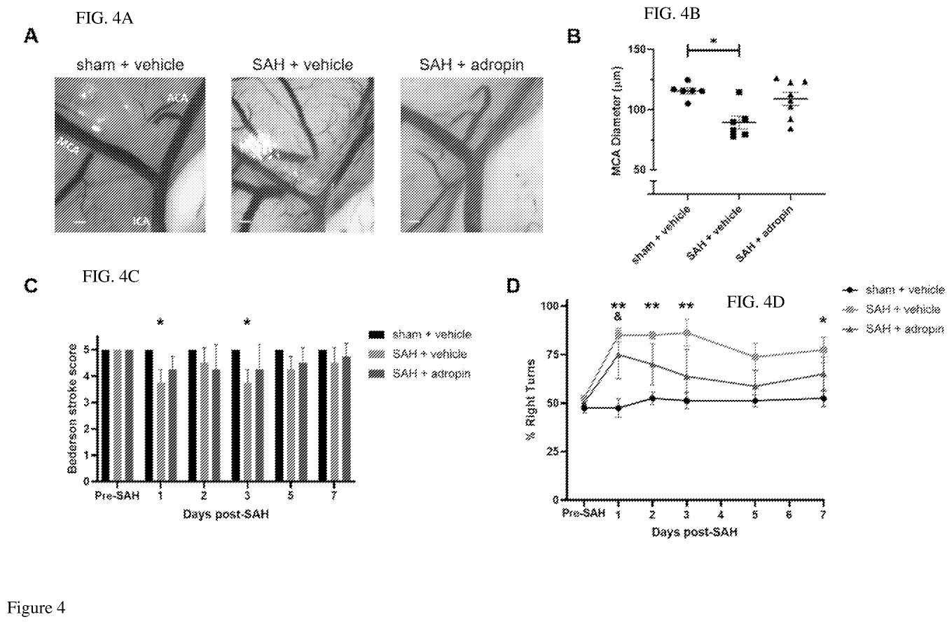

Delayed cerebral vasospasm, defined as decreased lumen diameter of the main cerebral vessels, is frequently observed via angiography following SAH.²² As a result, the perfusion to the corresponding cerebrum may be diminished, causing ischemic changed and eventually, functional deficits. The effect of adropin on cerebral vasospasm was investigated by measuring ipsilateral MCA diameter at seven days post-SAH. It was observed that SAH+vehicle treatment induced ˜20% reduction in vessel diameter compared to sham and that adropin-treatment reversed this phenotype (FIG. 4A&B). The behavior and functional status of these mice was analyzed to determine the effect of adropin treatment on the development of functional neurological deficits. It was found that the SAH model induces mild neurological dysfunction detected by the Bederson stroke scale²³ (FIG. 4C); however, a clear difference was observed in corner turn preference, which is more specific for a deficit created by right-sided SAH and MCA vasospasm. Adropin treatment is capable of reducing this sensorimotor deficit and normalizing corner turn preference (FIG. 4D).

### Example 6

### Neuronal Apoptosis is Reduced by Adropin Treatment after SAH

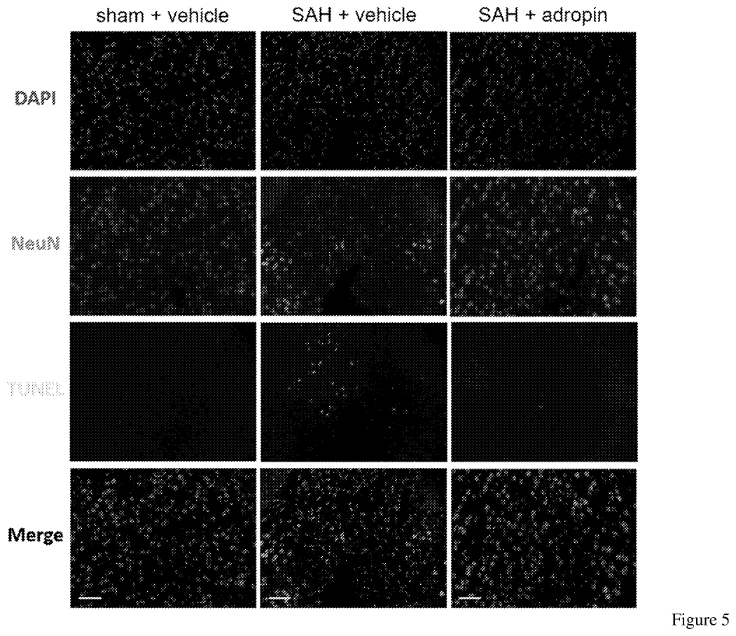

Neuronal apoptosis directly contributes to the neurological deficits observed in aSAH patients suffering from DCI. To evaluate the effect of adropin on apoptosis in the delayed phase of SAH pathology, we performed TUNEL staining at seven days post-SAH. An increased number of NeuN⁺/TUNEL⁺ cells were observed in the SAH+vehicle group compared to sham. Adropin-treated mice did not have as many TUNEL⁺ neurons at one-week post-SAH (FIG. 5).

### Example 7

### Adropin Promotes eNOS Pathway Activation at 1 & 7-Days Post-SAH

Nitric oxide is a critical regulator of endothelial function and vasodilation.²⁴ eNOS dysfunction after SAH is a well-studied phenomenon and known to contribute to brain injury.^{4,25} The effect of adropin on eNOS expression and activation was investigated by performing western blotting against total eNOS and p-eNOS (Ser1179).

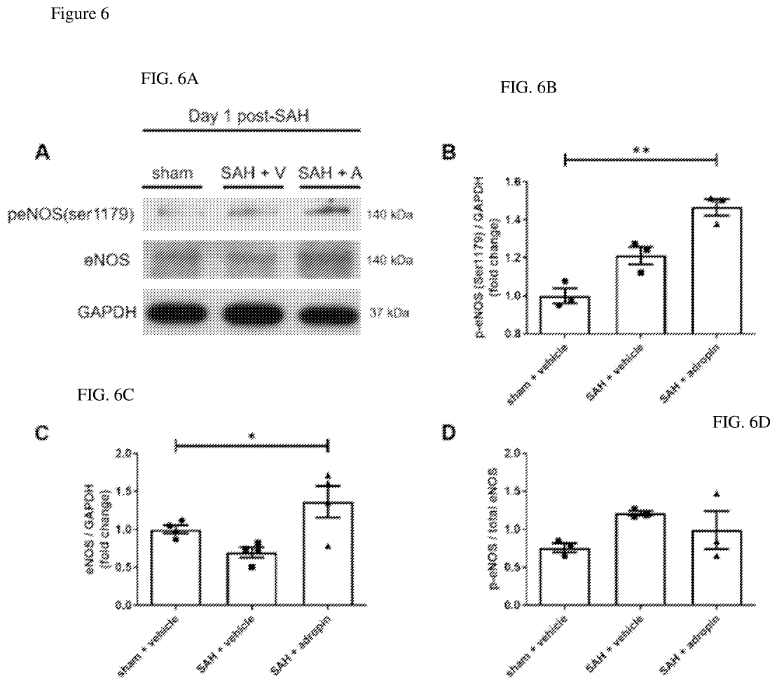

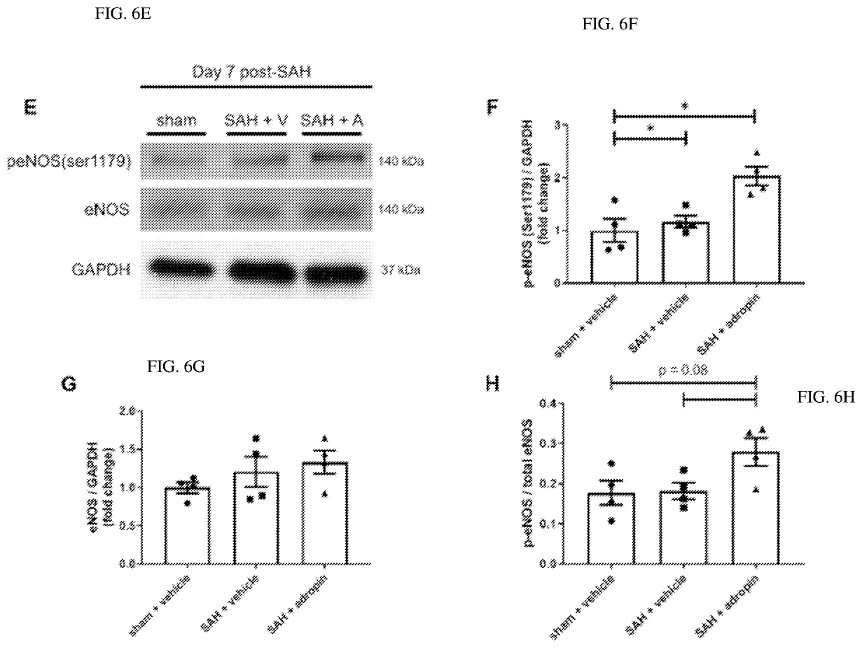

Treatment with exogenous adropin increased eNOS phosphorylation one day post-SAH as well as total eNOS compared to SAH+vehicle (FIG. 6A). There was no change in p-eNOS/total eNOS ratios between any groups (FIG. 6A), likely influenced by the decrease in total eNOS expression in the SAH+vehicle group. Loss of NO bioavailability has been demonstrated to affect both the early and delayed phases of SAH pathology, so the eNOS pathway at the 7 day post-SAH timepoint was investigated to further delineate adropin's effect on nitric oxide production. Similar to the acute phase, adropin treatment increase phospho-eNOS levels. Total eNOS was also slightly increased in both SAH groups; however, neither reached statistical significance. Phospho-eNOS/eNOS ratio trended higher with adropin treatment as well (p=0.08 vs SAH+vehicle, p=0.08 vs sham+vehicle) (FIG. 6B). The results indicate adropin mediation of the eNOS pathway persists throughout various phases of SAH pathology.

### Example 8

### Adropin has No Effect after SAH in Mice Lacking eNOS

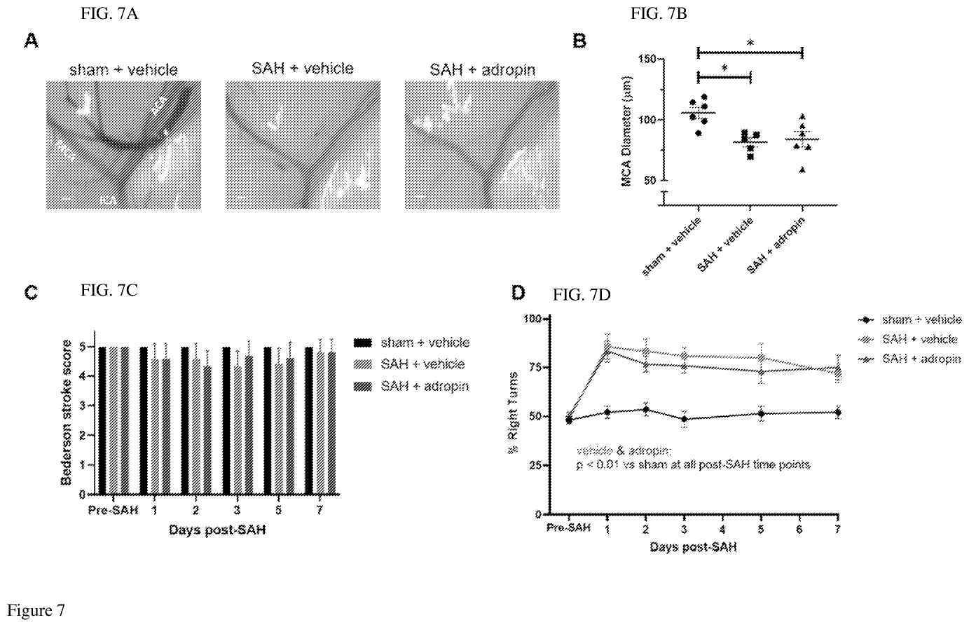

Given adropin's effects on the eNOS pathway and previous studies of the functional importance of NO signaling after SAH, it was hypothesized that adropin's beneficial effect requires the activity of eNOS. To test this hypothesis, SAH was induced in mice with genetic disruption of eNOS (eNOS^{-/-}) and randomized them to receive either vehicle or adropin treatment. It was found that eNOS^{-/-} mice developed vasospasm similar to wild-type mice and that adropin did not prevent vasospasm (FIG. 7A&B). Functionally, adropin also had no effect on sensorimotor deficits. eNOS^{-/-} mice did not develop major deficits as measured by the composite neurological score, but showed increased turn preference in the corner test after SAH regardless of adropin treatment (FIG. 7C&D). These findings support the hypothesis that adropin acts through the eNOS pathway to exert its beneficial effects.

### Example 9

### The Protective Effect of Adropin is Preserved if Treatment Initiation is Delayed Until 24 Hours Post-SAH

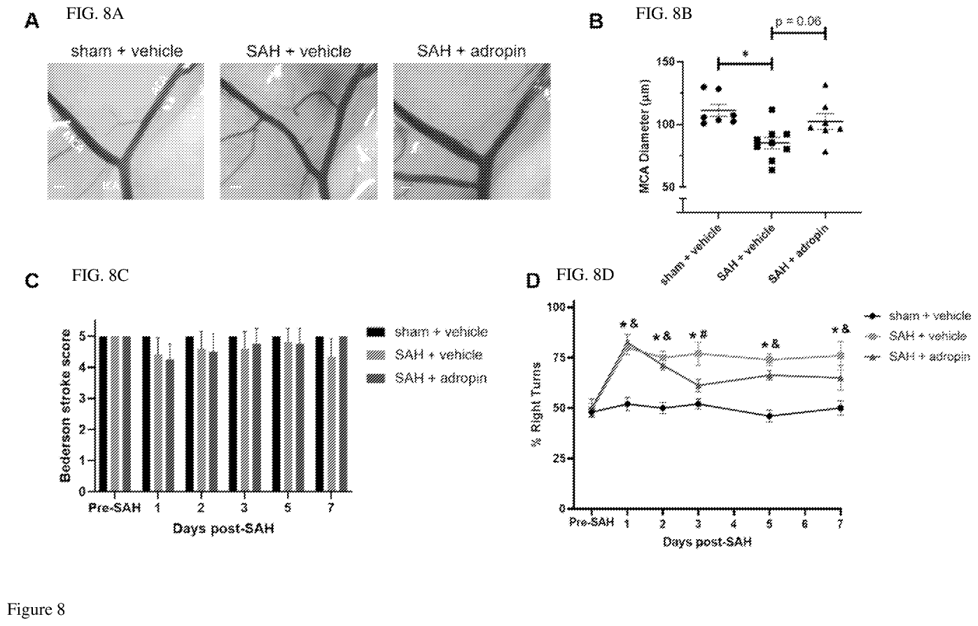

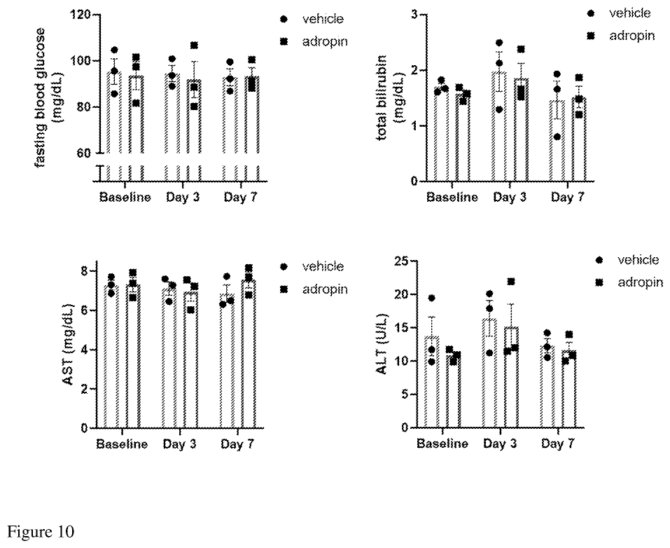

Translational investigation of any therapeutic requires careful consideration of its incorporation into current clinical standards of care. For patients presenting with aSAH, the most immediate priority is attaining hemostasis through securing the culprit aneurysm. Treatments to reduce secondary brain injury, especially those which may impact hemodynamics, are likely to be deferred until after surgical/endovascular interventions. The effect of delayed adropin treatment was tested by initiating treatment at 24 hours post-SAH instead of 15 minutes post-SAH. The ability of adropin treatment to improve sensorimotor deficits was maintained under this treatment paradigm and there was a strong trend for reduced cerebral vasospasm as well (FIG. 8A-D). Separately, there has been some speculation that supraphysiological doses of adropin may be hepatotoxic since adropin is known to regulation liver metabolism. Quantitative assays were conducted for common markers of liver toxicity and found that adropin had no effect compared to vehicle treatment (FIG. 10). These data suggest that adropin could be well-suited for clinical testing and may avoid some of the common pitfalls of novel therapeutics.

### Example 10

### Adropin Treatment Improves Outcomes in Murine Intracranial Aneurysm Rupture Model

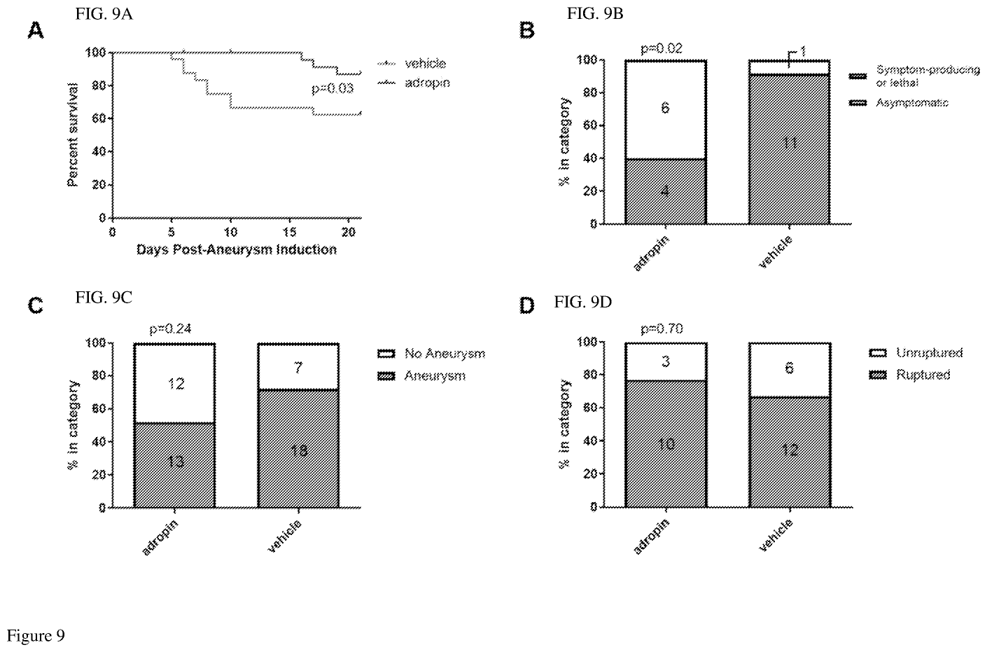

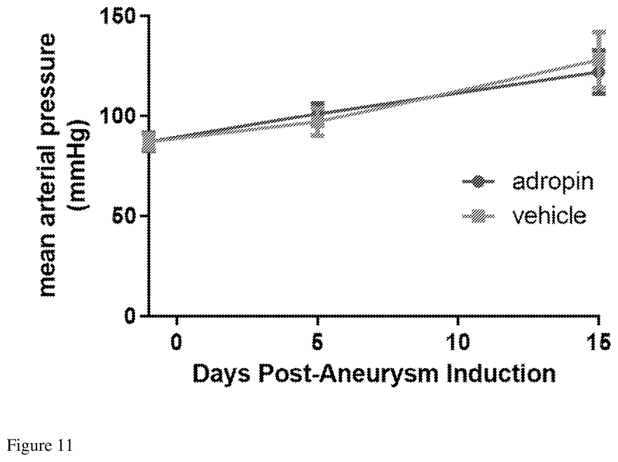

Given the beneficial effects of adropin in the SAH model, adropin treatment was tested in an aSAH-specific murine model. The presence of an aneurysmal change and vascular inflammation prior to subarachnoid hemorrhage distinguishes aneurysmal SAH from other forms of hemorrhagic stroke and SAH. Adropin-treated animals exhibited extended deficit-free survival compared to vehicle (FIG. 9A). Further, aneurysm ruptures in adropin-treated animals were less likely to be symptom-producing (mice did not exhibit decreased motor activity, weight loss, or circling paresis and evidence of rupture was only found upon autopsy at the end of the study) compared to vehicle-treated animals (FIG. 9B). It was found that adropin treatment did not significantly reduce aneurysm formation (FIG. 9C) or aneurysm rupture (FIG. 9D). Systemic blood pressure is an important factor in cerebral aneurysm models that could potentially cloud the interpretation of the results. No difference was found in blood pressure between vehicle-treated and adropin-treated mice in the aneurysm rupture model (FIG. 11).

### REFERENCES

1. Geraghty, J. R., Davis, J. L. & Testai, F. D. Neuroinflammation and Microvascular Dysfunction After Experimental Subarachnoid Hemorrhage: Emerging Components of Early Brain Injury Related to Outcome. Neurocrit. Care (2019) doi:10.1007/s12028-019-00710-x.
2. Pluta, R. M. et al. Loss of nitric oxide synthase immunoreactivity in cerebral vasospasm. J. Neurosurg. 84, 648-654 (1996).
3. Pluta, R. Delayed cerebral vasospasm and nitric oxide: review, new hypothesis, and proposed treatment. Pharmacol. Ther. 105, 23-56 (2005).
4. Pluta, R. M. Dysfunction of nitric oxide synthases as a cause and therapeutic target in delayed cerebral vasospasm after SAH. Acta Neurochir. Suppl. 104, 139-47 (2008).
5. Vergouwen, M. DI, Vermeulen, M., Coert, B. A., Stroes, E. S. & Roos, Y. B. Microthrombosis after Aneurysmal Subarachnoid Hemorrhage: An Additional Explanation for Delayed Cerebral Ischemia. J. Cereb. Blood Flow Metab. 28, 1761-1770 (2008).
6. Peeyush Kumar, T. et al. Endothelial Cell Dysfunction and Injury in Subarachnoid Hemorrhage. Mol. Neurobiol. 56, 1992-2006 (2019).
7. Velat, G. J., Kimball, M. M., Mocco, J. D. & Hoh, B. L. Vasospasm After Aneurysmal Subarachnoid Hemorrhage: Review of Randomized Controlled Trials and Meta-Analyses in the Literature. World Neurosurg. 76, 446-454 (2011).
8. Kumar, K. G. et al. Identification of Adropin as a Secreted Factor Linking Dietary Macronutrient Intake with Energy Homeostasis and Lipid Metabolism. Cell Metab. 8, 468-481 (2008).
9. Lovren, F. et al. Adropin is a novel regulator of endothelial function. Circulation 122, S185-S192 (2010).
10. Yang, C., DeMars, K. M., Hawkins, K. E. & Candelario-Jalil, E. Adropin reduces paracellular permeability of rat brain endothelial cells exposed to ischemia-like conditions. Peptides 81, 29-37 (2016).
11. Yu, L. et al. Adropin preserves the blood-brain barrier through a Notch1/Hes1 pathway after intracerebral hemorrhage in mice. J. Neurochem. 143, 750-760 (2017).
12. Wu, C. W. et al. RNA processing errors triggered by cadmium and integrator complex disruption are signals for environmental stress. BMC Biol. 17, (2019).
13. Yang, C., DeMars, K. M. & Candelario-Jalil, E. Age-Dependent Decrease in Adropin is Associated with Reduced Levels of Endothelial Nitric Oxide Synthase and Increased Oxidative Stress in the Rat Brain. Aging Dis. 9, 322 (2018).
14. Yang, C., Hawkins, K. E., Doré, S. & Candelario-Jalil, E. Neuroinflammatory mechanisms of blood-brain barrier damage in ischemic stroke. Am. J. Physiol.—Cell Physiol. 316, C135-C153 (2019).
15. Candelario-Jalil, E., Yang, Y. & Rosenberg, G. A. Diverse roles of matrix metalloproteinases and tissue inhibitors of metalloproteinases in neuroinflammation and cerebral ischemia. Neuroscience vol. 158 983-994 (2009).
16. Claassen, J. et al. Global cerebral edema after subarachnoid hemorrhage: frequency, predictors, and impact on outcome. Stroke 33, 1225-32 (2002).
17. Lublinsky, S. et al. Early blood-brain barrier dysfunction predicts neurological outcome following aneurysmal subarachnoid hemorrhage. EBioMedicine 43, 460-472 (2019).
18. Saunders, N. R., Dziegielewska, K. M., Møllgård, K. & Habgood, M. D. Markers for blood-brain barrier integrity: how appropriate is Evans blue in the twenty-first century and what are the alternatives? Front. Neurosci. 9, 385 (2015).
19. Friedrich, B., Müller, F., Feiler, S., Schöller, K. & Plesnila, N. Experimental Subarachnoid Hemorrhage Causes Early and Long-Lasting Microarterial Constriction and Microthrombosis: An in-vivo Microscopy Study:
   http://dx.doi.org/10.1038/jcbfm.2011.154 (2011) doi:10.1038/JCBFM.2011.154.
20. Pennings, F. A., Bouma, G. J. & Ince, C. Direct Observation of the Human Cerebral Microcirculation During Aneurysm Surgery Reveals Increased Arteriolar Contractility. Stroke 35, 1284-1288 (2004).
21. Terpolilli, N. A., Brem, C., Bühler, D. & Plesnila, N. Are We Barking Up the Wrong Vessels? Stroke 46, 3014-3019 (2015).
22. Kassell, N. F., Sasaki, T., Colohan, A. & Nazar, G. Cerebral vasospasm following aneurysmal subarachnoid hemorrhage. Stroke 16, 562-572 (1985).
23. Bederson, J. B. et al. Rat middle cerebral artery occlusion: evaluation of the model and development of a neurologic examination. Stroke 17, 472-476 (1986).
24. Förstermann, U. & Münzel, T. Endothelial Nitric Oxide Synthase in Vascular Disease: From Marvel to Menace. Circulation 113, 1708-1714 (2006).
25. Sabri, M. et al. Uncoupling of Endothelial Nitric Oxide Synthase after Experimental Subarachnoid Hemorrhage. J. Cereb. Blood Flow Metab. 31, 190-199 (2011).
26. Tejero, J., Shiva, S. & Gladwin, M. T. Sources of vascular nitric oxide and reactive oxygen species and their regulation. Physiological Reviews vol. 99 311-379 (2019).
27. Sabri, M., Ai, J., Lass, E., D'abbondanza, J. & Macdonald, R. L. Genetic elimination of eNOS reduces secondary complications of experimental subarachnoid hemorrhage. J. Cereb. Blood Flow Metab. 33, 1008-14 (2013).
28. Vellimana, A. K. et al. Endothelial nitric oxide synthase mediates endogenous protection against subarachnoid hemorrhage-induced cerebral vasospasm. Stroke 42, 776-82 (2011).
29. Hoh, B. L. et al. Estrogen Deficiency Promotes Cerebral Aneurysm Rupture by Upregulation of Th17 Cells and Interleukin-17A Which Downregulates E-Cadherin. J. Am. Heart Assoc. 7, e008863 (2018).
30. Wajima, D. et al. Interleukin-6 Promotes Murine Estrogen Deficiency-Associated Cerebral Aneurysm Rupture. Neurosurgery 0, 1-10 (2019).
31. Hosaka, K., Downes, D. P., Nowicki, K. W. & Hoh, B. L. Modified murine intracranial aneurysm model: aneurysm formation and rupture by elastase and hypertension. J. Neurointerv. Surg. 6, 474-479 (2014).
32. Turan, N. et al. Sex Differences in the Formation of Intracranial Aneurysms and Incidence and Outcome of Subarachnoid Hemorrhage: Review of Experimental and Human Studies. Translational Stroke Research vol. 7 12-19 (2016).
33. Sabri, M. et al. Anterior circulation mouse model of subarachnoid hemorrhage. Brain Res. 1295, 179-185 (2009).
34. Keep, R. F., Hua, Y. & Xi, G. Brain water content. A misunderstood measurement? Transl. Stroke Res. 3, 263-5 (2012).
35. Natah, S. S., Mouihate, A., Pittman, Q. J. & Sharkey, K. A. Disruption of the blood-brain barrier during TNBS colitis. Neurogastroenterol. Motil. 17, 433-446 (2005).
36. Vellimana, A. K. et al. Minocycline protects against delayed cerebral ischemia after subarachnoid hemorrhage via matrix metalloproteinase-9 inhibition. Ann. Clin. Transl. Neurol. 4, 865-876 (2017).
37. Takemoto, Y. et al. The Stabilization of Central Sympathetic Nerve Activation by Renal Denervation Prevents Cerebral Vasospasm after Subarachnoid Hemorrhage in Rats. Transl. Stroke Res. 11, 528-540 (2020).
38. Janssen, B., Debets, J., Leenders, P. & Smits, J. Chronic measurement of cardiac output in conscious mice. Am. J. Physiol.—Regul. Integr. Comp. Physiol. 282, (2002).

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are described. All publications mentioned herein, are incorporated by reference in their entirety. Nothing herein is to be construed as an admission that the invention is not entitled to antedate such disclosure by virtue of prior invention.

What is claimed is:

1. A method for treating a brain condition in a subject in need comprising administering an therapeutically effective amount of adropin, or variants thereof, and a pharmaceutically acceptable carrier.

2. The method of claim 1, wherein administering comprises a dose of 90 nmol/kg to 900 nmol/kg.

3. The method of claim 2, wherein the adropin is in a composition that comprises a dose of about 450 nmol/kg.

4. The method of claim 1, wherein administering comprises a dose of 0.1 mg to 5 g.

5. The method of any of claims 1-4, the brain condition comprises brain hemorrhage, intracerebral hemorrhage, subarachnoid hemorrhage (SAH), cerebral edema, blood brain barrier (BBB) damage, vasospasm, or sensorimotor deficits.

6. The method of claim 5, wherein the brain condition is SAH.

7. The method of any of claim 6, wherein the adropin is first administered 15 minutes to 24 hours after a SAH.

8. The method of claim 6, wherein the adropin is administered every other day after a SAH for a period of from 0.5 to 3 weeks.

9. The method of any of claims 1-8, wherein the adropin effectively increases the level of endothelial nitric oxide synthase (eNOS) in a subject in need.

10. The method of claim 1, wherein the administering comprises parenteral, intracranial, intraperitoneal, subcutaneous, intramuscular, or intravenous injection.

11. A method of reducing the risk of or treating vasospasm in a subject having subarachnoid hemorrhage (SAH) comprising administering a therapeutically effective amount of a composition comprising adropin, or variants thereof, and optionally a pharmaceutically acceptable carrier.

12. The method of claim 11, wherein the composition is administered 15 minutes to 24 hours after a SAH.

13. The method of claim 11 or 12, wherein administering is conducted according to a regimen of 1-5 doses daily or every other day for 0.5 to 4 weeks.

14. The method of any of claims 11-13, wherein administering comprises parenteral, intracranial, intraperitoneal, subcutaneous, intramuscular, or intravenous injection mode of administration.

15. The method of any of claims 11-13, wherein administering comprises oral administration.
